(19) United States
(12) Patent Application Publication
(10) Pub. No.: US 2022/0139255 A1
(43) Pub. Date: May 5, 2022

(54) DEVICES, SYSTEMS, AND METHODS FOR VOICE THERAPY

(71) Applicant: Innovative Voice Sciences and Strategies, LLC, Herriman, UT (US)

(72) Inventor: Matthew Keith Frank, Herriman, UT (US)

(21) Appl. No.: 17/086,214

(22) Filed: Oct. 30, 2020

(51) Int. Cl. G09B 19/04; A61M 16/06; A61B 5/00

(52) U.S. Cl. CPC G09B 19/04; A61B 5/4803; A61M 16/06

(57) ABSTRACT

A voice therapy mask includes a base that is configured to create a seal against a user's face and a body that forms an air pocket. A positive end-expiratory pressure (PEEP) valve is connected to the body to create a back-pressure in the user's lungs and vocal cords. The mask allows the user to move his or her mouth to perform speech therapy exercises, thereby increasing the range of available vocal cord strengthening exercises.

### CROSS-REFERENCE TO RELATED APPLICATIONS

N/A.

### BACKGROUND

### Background and Relevant Art

Humans vocalization is a behavior that involves a complex relationship between the vibratory source (e.g., the vocal cords or vocal folds) and the filter (e.g., the bifurcated vocal tract which is composed of the pharynx or throat, the oral cavity, and the nasal cavity). Vocalization is achieved when the air passed up from the lungs sets the vocal folds into vibration. A pressure wave is created as the vocal folds vibrate in this stream of air with the subsequent acoustic features being shaped by the vocal tract. Voice can be altered by changing factors that include but are not limited to the elasticity and shape of the vocal folds, the volume of air passed up from the lungs, and constriction of the vocal tract. Changes may be subjectively perceived (e.g., timbre or resonance), acoustically measured (e.g., cepstrum profile or vocal intensity measured in decibels), or visually defined using clinical instruments (e.g., mirror exam or videolaryngostroboscopy).

Strength plays a role in both objective and subjective measures of voice. Strength involves flexibility, mobility (i.e., power through the flexibility range), and stability. Like any other muscles, the vocal folds may be strengthened through appropriate use and voice therapy. Conversely, through disuse, disease, trauma, and other factors, the vocal folds may become weaker. Weakening of the vocal folds results in changes to the subjective perception, associated acoustics, and the clinical behavior of the voice.

### BRIEF SUMMARY

In some embodiments, a voice therapy device includes a voice therapy mask that is configured to provide a seal around a user's mouth and nose. The voice therapy mask has an opening and allows the user's mouth to open. The voice therapy device further includes a positive end-expiratory pressure (PEEP) valve connected to the voice therapy mask at the opening. The PEEP valve is configured to allow for an air pressure impedance in a body of the.

In other embodiments, a method for voice therapy includes applying a voice therapy mask to a user's face. The voice therapy mask is configured to cover the user's mouth and nose. A PEEP valve connected to the voice therapy mask is set to an impedance level, and the voice therapy mask is pressed against the user's face until a seal is achieved. While the voice therapy mask is sealed against the user's face, a voice therapy protocol is performed. The voice therapy protocol includes producing a first kind of vocalization in the voice therapy mask, producing a second, different, kind of vocalization in the voice therapy mask, and producing natural language in the voice therapy mask. In some embodiments, the user is instructed to perform the method by a speech-language pathologist and/or a medical professional.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1-1 and FIG. 1-2 are representations of a voice therapy mask, according to at least one embodiment of the present disclosure;

FIG. 2 is a representation of a user wearing a voice therapy mask, according to at least one embodiment of the present disclosure;

FIG. 3 is a representation of a method for speech therapy using a voice therapy mask, according to at least one embodiment of the present disclosure;

FIG. 4 is a representation of another method for speech therapy using a voice therapy mask, according to at least one embodiment of the present disclosure; and

FIG. 5 is a representation of a hydration protocol, according to at least one embodiment of the present disclosure.

### DETAILED DESCRIPTION

This disclosure generally relates to devices, systems, kits, and methods for speech therapy. Specifically, the devices, systems, kits, and methods described herein utilize a standard anesthesia mask with a connected adjustable positive end-expiratory pressure (PEEP) valve to provide back pressure to the vocal folds while breathing and/or vocalizing (e.g., speaking, singing, intoning). Vocalizing while wearing the voice therapy mask may change the vibratory behavior of the vocal folds during vocalization. Repeated vocalization while wearing the voice therapy mask may exercise the vocal cord muscles, causing them to strengthen over time.

A person may desire to change perceptual and acoustic (e.g., pitch and/or volume) features of his or her voice. For example, a person may be a performer, and wish to sing with an increased pitch range, an increased volume, and/or a different intonation. Furthermore, through disease, trauma, or disuse, a person may have a reduced vocal cord capability. A speech-language pathologist (e.g., a speech therapist) or other healthcare provider may work with a user to strengthen his or her vocal cords. The speech-language pathologist may instruct the user to perform exercises, such as vocalizing certain sounds in a certain order, vocalizing with a specific pitch, vocalizing with a specific volume, and/or any other exercise.

Conventionally, speech-language pathologists may instruct the user to perform exercises with an increased support of the voice from the respiratory system. For example, conventional voice therapy devices may include a hollow tube, such as a straw, to be held in the user's mouth. The user may vocalize with the straw in his mouth, which may increase the pressure on the lungs and vocal cords of the user. However, because the device is held in the user's mouth, the user does not have a full range of motion of his mouth, and therefore is limited in the sounds that he or she can make. Furthermore, such systems may not be able to provide sufficient back pressure to strengthen the vocal cord muscles. Other conventional voice therapy programs may attempt to create a back pressure by pursing and/or protruding the lips while vocalizing at specific pitches, which may similarly not be able to provide sufficient back pressure to strengthen the vocal cord muscles.

In accordance with embodiments of the present disclosure, a user may place a mask over her nose and mouth. The mask may have an opening which may be partially or fully closed to increase the impedance inside a body of the mask, resulting in a back pressure on the vocal cords. Because the nose and mouth are covered by the mask, the user is free to vocalize normally. This approach may allow for therapeutic benefits to generalize to normal speech and singing.

As discussed above, conventional devices are held in the mouth. Thus, if a user opens his mouth to make a certain sound, the conventional device may fall out of his mouth and/or limit movement of the lips, jaw, and tongue which are used to articulate speech. Pursing and/or protruding of the lips may similarly limit movement of the lips, jaw, and tongue. This may reduce the effectiveness of conventional devices and programs by limiting the types of sounds that the user can make. In accordance with embodiments of the present disclosure, because the mask is placed completely over the mouth of the user, the user may move and/or open his mouth to vocalize. This may increase the range of sounds available to be made by the user.

As will be discussed further herein, a valve may be placed in a hole in the mask. The valve may provide back-pressure to the user's larynx and lungs. For example, the valve may be set to an impedance level. As the user exhales (either while breathing or by vocalizing), the pressure in the mask may increase and be redirected as back pressure onto the vocal folds and further down the respiratory system into the lungs. While the pressure in the mask is at the impedance level set on the PEEP valve, voicing may be efficiently maintained, and there may not be an excess of air leaking through the PEEP valve. If the impedance level is not met, then voicing may not be produced as the back pressure on the larynx and lungs may push the vocal folds to an open position. If the user is using excessive effort, then the impedance is overcome and the vocal folds may be forcefully contracted.

By creating a back-pressure on the user's vocal folds and lungs, the vibratory behavior of the vocal folds may be changed. By vocalizing while subject to the back-pressure, the vocal cords may be strengthened and/or shaped. In accordance with embodiments of the present disclosure, the back-pressure caused by the valve may be variable. This allows for healthy adaptation under progressive overload of the voice. Progressive overload may encourage neuromuscular adaptation of the vocal folds, where the brain recruits progressively more muscular effort in a controlled manner that is safe for the patient. This variability may help the user to improve the quality, functional use, and objective measures of the voice.

Using a PEEP valve in conjunction with an anesthesia mask may result in back pressure or air pressure directed down the throat and onto the vocal folds. Back pressure pushes the vocal folds apart (e.g., abduction). This may result in greater flexibility of the vocal folds and greater mobility of the vocal folds as natural pitch variation in speech or singing is targeted with the treatment protocol. Because opened vocal folds contract and touch for voice, greater stability of the vocal folds may also be achieved. Furthermore, and as discussed herein an adjustable PEEP valve allows for progressive overload over the course of treatment which provides therapeutic adaptation for users as physiologic challenges are sequentially overcome. Because the PEEP valve impedes airflow which must be matched, users may also be presented with the immediate feedback for their effort level.

In accordance with embodiments of the present disclosure, the valve connected to the voice therapy mask may include any type of valve. For example, the valve connected to the voice therapy mask may include an adjustable positive end-expiratory pressure (PEEP) mask. In some embodiments, the valve may have a set impedance level. In some embodiments, the valve may have a variable impedance level.

In accordance with embodiments of the present disclosure, the voice therapy mask may be any type of mask. For example, the voice therapy mask may be a standard medical anesthesia mask. In some examples, the voice therapy mask may be a standard medical oxygen mask.

In accordance with embodiments of the present disclosure, a vocalization is any sound created by a user by vibrating the vocal cords. Vocalizations may include any sounds, including speaking, reading, singing, yelling, whispering, any other sound, and combinations thereof. In some embodiments, a vocalization may be a single phoneme (e.g., a single unit of sound used in combination to form words). In some embodiments, a vocalization may include multiple phonemes strung together. In some embodiments, a vocalization may include a vowel sound. In some embodiments, a vocalization may include a consonant sound. In some embodiments, different vocalizations may include variations in pitch and/or volume of the same phonemes and/or words.

In accordance with embodiments of the present disclosure, a speech-language pathologist or other medical professional may provide a user with a speech therapy treatment to perform while wearing the voice therapy mask. The speech therapy treatment may include a series of discrete exercises for the user to perform while wearing the mask. For example, the speech therapy treatment may include instructing the user to hold a certain vocalization for a period of time. The vocalization may be any vocalization, such as “ah,” “oh,” “ee,” “mm,” or any other vocalization. In some embodiments, the speech therapy treatment may include instructing the user to change the pitch of the vocalization. For example, the speech therapy treatment may include instructing the user to begin vocalizing at a first pitch (e.g., the note F below middle C), and to smoothly change the pitch (e.g., glide) to a pitch (e.g., a note) above or below the first pitch. In some embodiments, the speech therapy treatment may include instructing the user to change the volume of the vocalization. For example, the user may be instructed to begin vocalizing at a first volume and then to increase or decrease the volume of the vocalization. In some embodiments, the user may be instructed to change both the pitch and the volume while vocalizing.

In some embodiments, the speech therapy treatment may include a therapy protocol of discrete exercises to be performed while wearing the voice therapy mask. For example, a therapy protocol may include four exercises or steps in sequence with established repetitions. The four steps may include a constant vocalization, two varied vocalizations (e.g., a glide up and/or down in pitch), and one exercise of natural speech (e.g., singing, talking, reading). Each exercise in the therapy protocol may be repeated. For example, the first exercise may be performed twice before moving to the second exercise, which may be repeated twice before moving to the third exercise, and so forth. In some examples, the four exercises may be completed in order and then repeated in the same order or a different order (e.g., reverse order, or a mixed up order).

In some embodiments, the speech therapy treatment may include repeating the therapy protocols. For example, a therapy protocol may be performed daily for a period of time (e.g., a week, two weeks, a month). In some examples, the therapy protocol may be performed twice per day (e.g., once in the morning, and again in the afternoon/evening). In some examples, the therapy protocols may be performed in a clinical setting (e.g., at the speech-language pathologist's office). In some examples, the therapy protocols may be performed at home. In some embodiments, the speech-language pathologist may provide instructions for each therapy protocol in a clinical setting, and then send the voice therapy mask home with the user to perform the therapy protocols at home. In some embodiments, the user may meet with the speech-language pathologist regularly in the clinical setting and perform therapy protocol at home between meetings.

In some embodiments, the speech therapy treatment may include checking a user's hydration using a hydration protocol. The quality of a person's voice may be related to his or her hydration level. A low level of hydration may stiffen the vocal cords and cause them to lose some volume or size. This may lead to a greater exertion when vocalizing, leading to a higher likelihood of vocal cord injury and excessive contraction of the vocal cords and the vocal tract. Prior to performing a therapy protocol, a speech-language pathologist and/or medical professional may instruct a user to check his or her hydration. The user may check hydration using a capillary refill check. To perform a capillary refill check, the user may press his fingertips into the skin of his lower ribcage hard enough to cause the skin to blanch or lighten in color. After pulling his fingers away, the user may be instructed to watch the space of the pressure. If the tone or coloration returns to normal within 3 seconds, then hydration may be high enough to proceed with the therapy protocol. If not, the user may be instructed to drink more water and repeat the process later (such as 30 minutes). This process may be repeated until hydration levels are high enough to continue with the therapy.

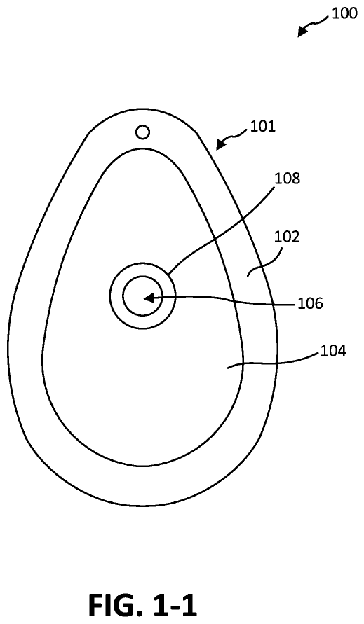

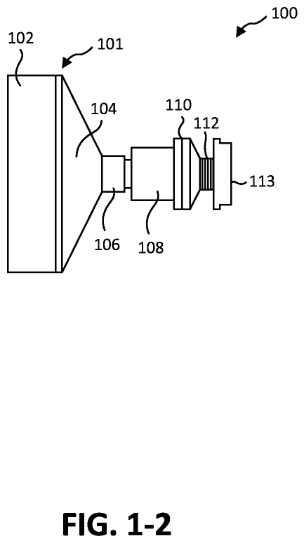

Referring now to the figures, FIG. 1-1 is a representation of a voice therapy system 100, according to at least one embodiment of the present disclosure. The voice therapy system 100 shown includes a mask 101 having a base 102 (e.g., a mask cushion) and a body 104. The base 102 may be formed from a flexible material that may conform to a user's face to form a seal. The body 104 may extend from the base and be formed from a hard material. The hard material of the body 104 may form a pocket of air between the user's mouth and the body 104. This may allow space for user to move her mouth while the voice therapy system 100 is placed against his or her face. In some embodiments, the hard material of the body 104 may be rigid, and may support a positive and/or a negative pressure in the pocket of air formed between the user's mouth and the body 104.

In some embodiments, the body 104 may include an aperture or opening 106. The opening 106 may be a port for air and other gasses to flow into and out of the pocket of air formed by the body 104. The opening 106 has an opening diameter, which may be the diameter of the port for air and other gasses to flow into and out of the pocket of air. In some embodiments, a valve 108, such as a PEEP valve, may be placed in the opening 106. In some embodiments, the valve 108 may be a bi-direction valve. In some embodiments, the valve 108 may be a unidirectional valve. In other words, the valve 108 may allow air to leave the body 104, but may not allow air to enter into the body 104. In some embodiments, impedance of air pressure at the valve 108 may create an excess of positive pressure under the body 104 and result in re-direction of air pressure to the vocal folds and/or lungs of the user. A positive pressure inside the body 104 may cause air to flow out of the body 104.

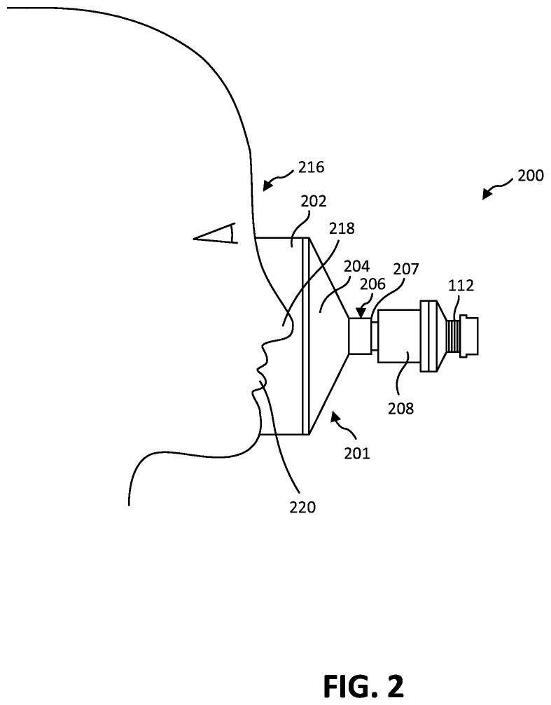

FIG. 1-2 is a representation of a side-view of the voice therapy system 100 of FIG. 1-1, which includes a mask 101 and a valve 108. In some embodiments, the valve 108 may have an impedance level. At pressures below the impedance level, the valve 108 may prevent air pressure from flowing out of the body 104. For example, the valve 108 may include a diaphragm 110 (or other valve member) connected to a spring 112. The spring 112 may urge the diaphragm 110 to prevent air from leaving the body 104. The exhalatory pressure in the body 104 causes an opposing force against the diaphragm 110. When the pressure in body 104 pushes on the diaphragm 110 stronger than the spring 112 (e.g., when the pressure in the body 104 is greater than the impedance level), air may exit the body 104 through the valve 108. When the pressure in the body pushes on the diaphragm 110 less strongly than the spring 112 (e.g., when the pressure in the body 104 is less than or equal to the impedance level), the diaphragm 110 may close the valve 108, thereby impede air from exiting the valve.

In some embodiments, the impedance level may help to create a back pressure in the user's vocal tract down to the vocal folds and/or into the lungs while vocalizing. For example, the impedance level may represent a minimum force used to overcome the impedance of the valve 108 and move air out of the body 104 of the voice therapy system 100. The impedance level may represent an increase in pressure from the lungs and increased effort from the vocal folds when vocalizing while wearing the voice therapy mask. This may change the shape and/or vibration of the vocal cords. Performing one or more therapy protocols with the increased back-pressure may strengthen the vocal cords and help the user to improve the subjective features of voice quality, functional voice use, and objective measures of voice production.

In some embodiments, the impedance level may be in a range having an upper value, a lower value, or upper and lower values including any of 5 cm water (490 Pa), 10 cm water (981 Pa), 15 cm water (1.47 kPa), 20 cm water (1.96 kPa), 25 cm water (2.45 kPa), or any value therebetween. For example, the impedance level may be greater than 5 cm water (490 Pa). In another example, the impedance level may be less than 25 cm water (2.45 kPa). In yet other examples, the impedance level may be any value in a range between 5 mm water (490 Pa) and 25 cm water (2.45 kPa). In some embodiments, it may be critical that the impedance level is greater than or equal to 5 cm water (490 Pa) to provide back-pressure to strengthen the vocal cords. In some embodiments, the impedance level may be less than 5 cm water (49.0 Pa) or greater than 25 cm water (2.45 kPa).

In some embodiments, the impedance level of the valve 108 may be variable. For example, the valve 108 may include an adjustment mechanism 113. The adjustment mechanism 113 may change the pressure applied by the spring 112 to the diaphragm 110. This may allow the speech-language pathologist and/or health care provider to tailor and/or optimize the impedance level based on the circumstances and treatment plan of the user. In the embodiment shown, the adjustment mechanism 113 is an end-cap of the adjustable PEEP valve 208. By rotating the end-cap, the impedance level of the PEEP valve 208 may be increased or decreased. In some embodiments, the speech-language pathologist may utilize progressive overload to therapeutic effect.

FIG. 2 is a representation of a speech therapy system 200, including an adjustable PEEP valve 208 inserted into an opening 206 of a mask 201. The opening 206 of the mask may have a different diameter than the PEEP valve 208, and may utilize an adapter 207 to connect the PEEP valve 208 to the mask 201. In the embodiment shown, the mask 201 is attached to the face 216 of a user. The base 202 (e.g., cushion) of the mask 201 may be pressed up against the face 216 of the user. The base 202 may be formed from a flexible material that may conform to the face 216 of the user. To attach the voice therapy system 200 to the face 216 of the user, the user may place the base 202 over the user's face 216 so that the body 204 covers the nose 218 and the mouth 220 of the user. Because the base 202 is flexible, the user may push the base 202 against his face 216 until the base 202 forms a seal against the face 216. In the embodiment shown, the mask 201 is covering the user's mouth 220 and nose 218. However, it should be understood that the mask 201 may be configured to only cover the user's mouth.

The voice therapy system 200 includes a valve 208, such as an adjustable PEEP valve. The valve 208 may increase impedance of air pressure under the body 204 of the mask 201. The valve 208 may be set to a specific impedance level, as discussed herein. In some embodiments, the seal formed against the face 216 may be as strong as or stronger than the impedance level. In some embodiments, the seal may be strong enough to withstand the pressure of the user's breath that is exhaled while vocalizing. In some embodiments, the seal formed against the face 216 may help to prevent air from escaping the air pocket formed between the body 204 and the face 216. This may help to form the back pressure in the user's lungs.

In some embodiments, the mask 201 may allow the user to move her mouth 220 while vocalizing. For example, the air pocket formed between the body 204 and the face 216 may be large enough for the user's mouth 220 to move. Furthermore, the flexible material of the base 202 may provide freedom of movement for the user's mouth 220 to move. In some embodiments, the user's mouth 220 may be unencumbered during vocalization. In some embodiments, the user's mouth 220 may have a full range of motion for vocalizations. This may increase the range of vocalizations performed by the user. In some embodiments, this may allow the user to vocalize naturally, without any distortion or other changes to vocalizations caused by a limited range of movement of the mouth. In this manner, the user's vocal cords may be strengthened and/or their vocalizations otherwise improved.

In some embodiments, the user may maintain contact of the voice therapy system 200 against his or her face 216 by actively holding the assembled mask 201 and valve 208 and pushing the voice therapy mask against her face 216.

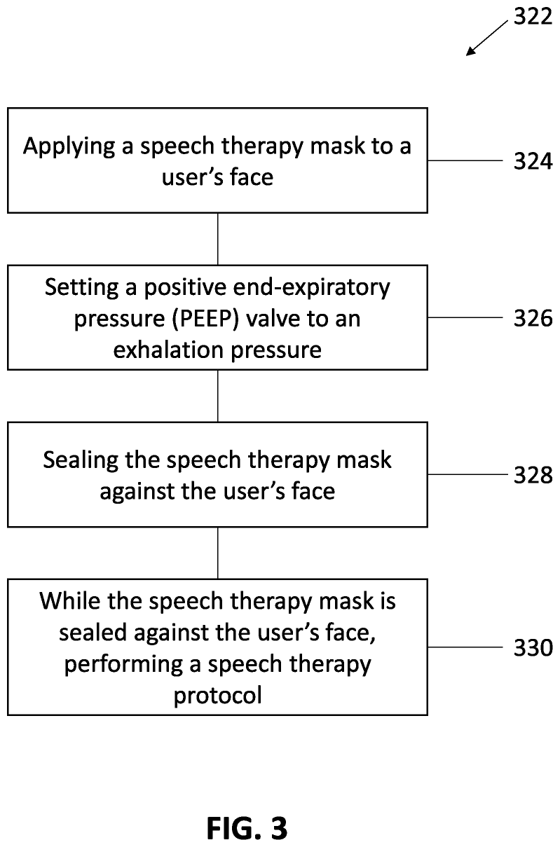

FIG. 3 is a representation of a method 322 for speech therapy, according to at least one embodiment of the present disclosure. In accordance with embodiments of the present disclosure, the method 322 may be performed by the voice therapy system 200 of FIG. 2. In other words, the voice therapy system 200 of FIG. 2 may be used to perform the speech therapy method 322 of FIG. 3.

The method 322 may include applying a voice therapy mask to a user's face at 324. The voice therapy mask may cover the user's mouth and nose. In some embodiments, the voice therapy mask may cover the user's mouth and nose such that the user may move his or her mouth to vocalize while wearing the voice therapy mask.

In some embodiments, the method 322 may include setting an adjustable positive end-expiratory pressure (PEEP) valve to an impedance level at 326. In some embodiments, the PEEP valve may be set to the impedance level before the user applies the voice therapy mask to the user's face. In some embodiments, the PEEP valve may be set to the impedance level after the user applies the voice therapy mask to her face.

In some embodiments, the method 322 may further include sealing the voice therapy mask against the user's face to form a seal capable of withstanding the impedance level at 328. While the voice therapy mask is sealed against the user's face, a therapy protocol may be performed at 330. In some embodiments, the therapy protocol may include performing one or more vocalizations. For example, the therapy protocol may include producing a first kind of vocalization while wearing the voice therapy mask. In some embodiments, the first kind of vocalization may be a first type of vocalization. For example, the first kind of vocalization may be a held vocalization, such as a single pitch that is held at a consistent volume for a vocalization period. In some embodiments, the single pitch is a high pitch in the user's voice range. In some examples, the first kind of vocalization may be repeated one, two, three, four, five, six, seven, eight, nine, ten, or more times.

In some examples, the therapy protocol may include producing a second kind of vocalization while wearing the voice therapy mask. The second kind of vocalization may be different from the first kind of vocalization. For example, the second kind of vocalization may be a variable vocalization, such as a glissando or glide from low to high pitch, high to low pitch, low to high volume, high to low volume, any other variable vocalization, and combinations thereof. In some embodiments, the second kind of vocalization may be held for a vocalization period. In some embodiments, the second kind of vocalization may be repeated one, two, three, four, five, six, seven, eight, nine, ten, or more times.

In some embodiments, the therapy protocol may include producing natural language (e.g., speech, singing, reading) while wearing the voice therapy mask.

In some embodiments, the method 322 may further include following a hydration protocol prior to performing the therapy protocol (e.g., prior to applying the voice therapy mask). The hydration protocol may include checking to see if the user's hydration levels are high enough to safely perform the therapy protocol.

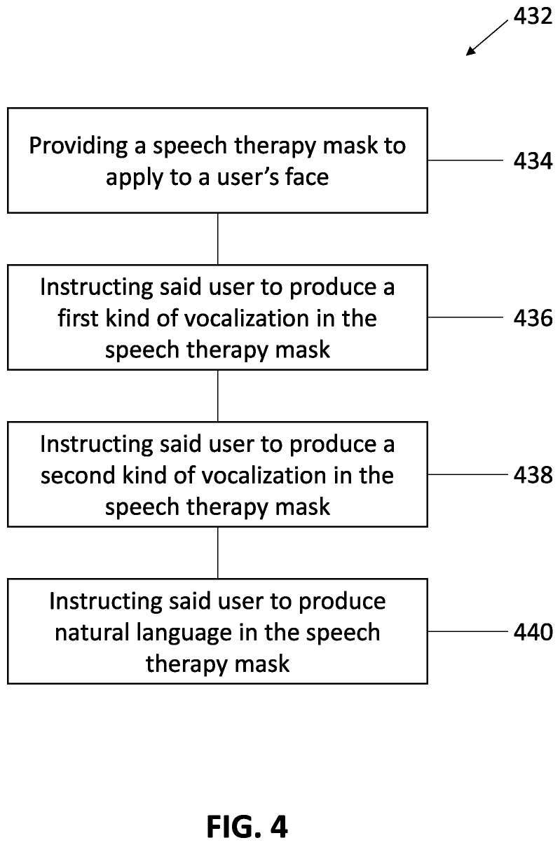

FIG. 4 is a representation of a method 432 for speech therapy, according to at least one embodiment of the present disclosure. In accordance with embodiments of the present disclosure, the method 432 may be performed by the speech therapy system 200 of FIG. 2. In other words, the voice therapy mask 200 of FIG. 2 may be used to perform the speech therapy method 432 of FIG. 4.

In some embodiments, the method 432 may include providing a voice therapy mask to a user's face at 434. The voice therapy mask may be provided by a speech-language pathologist or other medical professional. In some embodiments, the instructions to the user may include instructions to cover the user's mouth and nose to create a seal. The seal may be such that any exhaled air may only exit the voice therapy mask through a positive end-expiratory pressure (PEEP) valve connected to an opening in the mask. The PEEP valve may be set with an impedance level. The impedance level that may be maintained as the user produces vocalizations.

The method 432 may further include instructing the user to perform a therapy protocol. Instructing the user to perform the therapy protocol may include instructing the user to produce a first kind of vocalization into the voice therapy mask at 436. The first kind of vocalization may be any vocalization, such as those vocalizations discussed herein. In some embodiments, the first kind of vocalization may include vocalizing a single pitch and/or volume. In some embodiments, the first kind of vocalization may include a varying pitch and/or volume.

In some embodiments, the first kind of vocalization may be held for a vocalization duration. In some embodiments, the vocalization duration may be as long as possible (e.g., as long as the user can produce the vocalization without discomfort or severe discomfort before taking a breath and/or match the impedance of the PEEP valve). If a user's vocalizations cause excessive plosive air loss out of the PEEP valve, then the speech-language pathologist may make therapeutic adjustments to prevent the user from approaching hyper-functional behavior. In some embodiments, the vocalization duration may be in a range having an upper value, a lower value, or upper and lower values including any of 1 s, 5 s, 10 s, 15 s, 20 s, 30 s, 40 s, 45 s, 50 s, 1 min, 2 min, 3 min, 4 min, or any value therebetween. For example, the vocalization duration may be greater than 1 s. In another example, the vocalization duration may be less than 4 min. In yet other examples, the vocalization duration may be any value in a range between 1 s and 4 min. In some embodiments, it may be critical that the vocalization duration is as long as possible to strengthen the vocal cords without injuring the user. In some embodiments, the vocalization duration may be held without taking any intervening breaths. In some embodiments, the vocalization duration may be held until the user runs out of breath, and the user may then take a breath and continue the vocalization. In some embodiments, the vocalization duration may account for periods of time for a user to take the mask off to take a breath, and place the mask back on and continue vocalizing. In some embodiments, the vocalization duration may be different for men than for women. For example, a natural language vocalization duration may be 3.5 minutes for a woman and 4.0 minutes for a man.

In some embodiments, the speech-language pathologist or medical professional may instruct the user to produce a second kind of vocalization into the voice therapy mask at 438. The second kind of vocalization may be any vocalization, such as those vocalizations discussed herein. In some embodiments, the second kind of vocalization may be the same as the first kind of vocalization. In some embodiments, the second kind of vocalization may be different from the first kind of vocalization. In some embodiments, the second kind of vocalization may have a different pitch and/or volume than the first kind of vocalization. In some embodiments, the second kind of vocalization may include varying the pitch and/or volume while producing the vocalization. Making the second kind of vocalization different from the first kind of vocalization may help to exercise the vocal cords in different ways, which may further strengthen the vocal cords.

The speech-language pathologist or medical professional may further instruct the user to produce natural language into the voice therapy mask at 440. As discussed herein, natural language may include words, sentences, songs, poetry, books, dictation exercises, any other natural language, and combinations thereof. For example, the user may hold a conversation while wearing the mask. In some examples, the user may read a book or other written material. In some examples, the user may read poetry. In some embodiments, the user may perform dictation exercises, such as tongue twisters. In some examples, the user may perform vocal exercises, such as scales. In some examples, the user may sing. In some embodiments, the speech-language pathologist may instruct the user to produce the natural language for a vocalization duration. In some embodiments, the vocalization duration for producing natural language may be the same as the vocalization duration for producing the first/second kind of vocalization. In some embodiments, the vocalization duration for producing natural language may be shorter than for the first/second kind of vocalization. In some embodiments, the vocalization duration for producing natural language may be longer than for the first/second kind of vocalization. In some embodiments, in may be critical that the natural language vocalization duration is longer than the first/second kind of vocalization duration because it most closely represents how the user interacts with the world.

While embodiments of the methods of the present disclosure have been described with respect to two kinds of vocalization exercises and one natural language exercise, it should be understood that a therapy protocol may include more or fewer kinds of vocalization and/or natural language exercises. For example, a therapy protocol may include one or more kinds of vocalization exercises and no natural language exercise. In some examples, a therapy protocol may include one or more natural language exercises and no kinds of vocalization exercises. In some examples, a therapy protocol may include multiple kinds of vocalization exercises (e.g., two or more) and multiple natural language exercises. In some embodiments, the order of the exercises may be different than the order presented. For example, a natural language exercise may be performed first, followed by one or more kinds of vocalization exercises. In some examples, one or more kinds of vocalization exercises may be performed, followed by one or more natural language exercises, which may then be followed by one or more kinds of vocalization exercises. In this manner, a speech-language pathologist and/or medical professional may tailor a therapy protocol to the user. In some embodiments, different therapy protocols may have different combinations of exercises. For example, a therapy protocol to be performed in the morning may have a different combination of exercises than a therapy protocol to be performed in the afternoon/evening. In some embodiments, a speech-language pathologist or other medical professional may monitor the therapy protocols in a clinical session and change them based on the results of the clinical session.

In some embodiments, the healthcare provider may instruct the user to repeat one or more of the acts of the method 432 during a therapy protocol. For example, the healthcare provider may instruct the user to produce a first kind of vocalization one, two, three, four, five, six, seven, eight, nine, ten, or more times before producing the second kind of vocalization. Similarly, the healthcare provider may instruct the user to produce the second kind of vocalization one, two, three, four, five, six, seven, eight, nine, ten, or more times before producing natural language.

In some embodiments, the speech-language pathologist or the healthcare professional may instruct the user to rest between repetitions of an act, or to rest between acts in the therapy protocol. Resting between repetitions or acts may allow the user's vocal cord muscles to recover before exercising them again. Furthermore, resting between repetitions or acts may allow the user to adjust, such as engaging the diaphragm or breathing to prevent faintness.

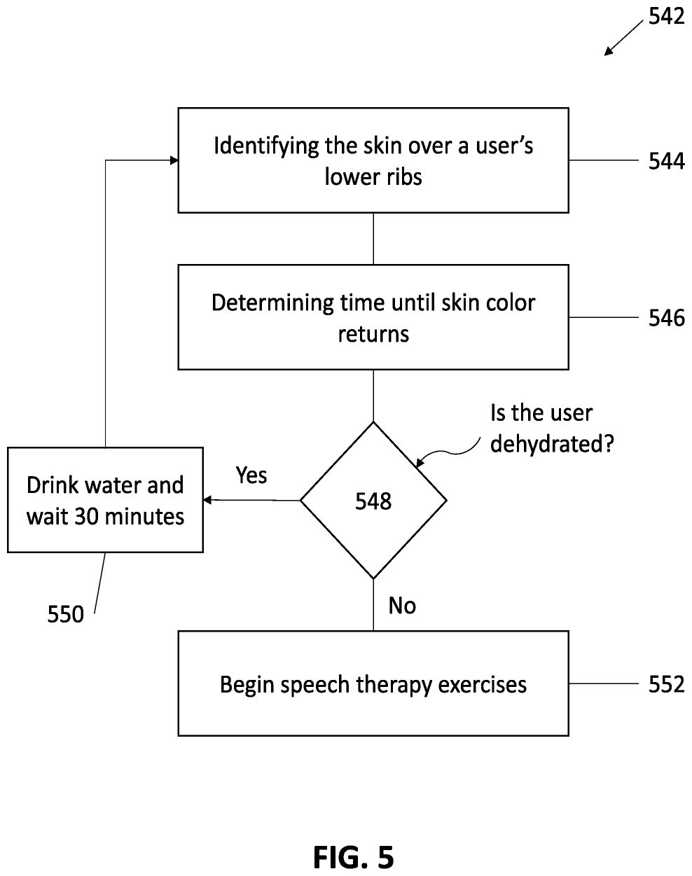

FIG. 5 is a representation of a hydration protocol 542 for checking a user's hydration before performing a speech therapy exercise and/or procedure, according to at least one embodiment of the present disclosure.

Hydration is important to a person's voice quality. As a person's hydration levels decrease, the vocal cords may stiffen and/or lose volume and/or size. This may result in a higher exertion and/or excessive effort when using his or her voice, which may increase the risk of injury. By checking a user's hydration prior to performing a speech therapy exercise and/or session, a user may help to reduce the chance of injury to the vocal cords caused by stiff and/or smaller vocal cords.

The hydration protocol 542 may include checking capillary refill. Capillaries are the small blood arteries and vessels at the surface of a person's skin. A user may identify a location to perform a hydration protocol, such as the skin over a user's lower ribs, at 544. The user may use his or her fingers to indent the skin. Indenting the skin may cause the blood to be pushed out of the capillaries. The user may indent his skin in any manner. For example, it may be easy to use his fingers to indent the skin. In other examples, the user may indent his skin using an implement. In some embodiments, the user may indent his skin on his torso. For example, the user may indent the skin on his chest near the bottom of the rib cage, such as at the lower ribs. The user may push on the skin hard enough to push the blood out of the capillaries. This may cause the skin to blanch, or to lighten in color. In some embodiments, the user may indent the skin at any location that may allow him to see a blanching or lightening in color of the skin.

The hydration protocol 542 may further include determining the amount of time until the user's skin color returns at 546. The amount of time it takes for the skin color to return (e.g., return to normal, return such that there is no difference in color between the depressed area of the skin and the surrounding skin) is the capillary refill time. In some embodiments, the capillary refill time may be an indication of the hydration of the user. If the capillary refill time is less than 3 seconds, then the user may be dehydrated. Based on the capillary refill time, the hydration protocol 542 may determine 548 whether the user is dehydrated. If the user is dehydrated, the hydration protocol 542 may instruct the user to drink water and wait thirty minutes at 550 before repeating the hydration protocol 542. If the user is not dehydrated, then the hydration protocol 542 is complete and the user may begin a speech therapy exercise and/or therapy protocol at 552. In some embodiments, a speech-language pathologist may instruct the user to perform the hydration protocol 542 before every therapy protocol to help reduce the chance of injury and/or avoid faintness.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A voice therapy device, comprising:
a voice therapy mask configured to provide a seal around a user's mouth and nose, the voice therapy mask having an opening, the voice therapy mask allowing said user's mouth to open; and
an adjustable positive end-expiratory pressure (PEEP) valve connected to the voice therapy mask at the opening, the PEEP valve being configured to retain an impedance level in a body of the voice therapy mask.

2. The device of claim 1, wherein the voice therapy mask is an anesthesia mask.

3. The device of claim 1, wherein the voice therapy mask allows said user's mouth to fully open.

4. The device of claim 1, wherein, while wearing the voice therapy mask, said user's mouth has a full range of motion for vocalizations.

5. A method for speech therapy, comprising:
applying a voice therapy mask to a user's face, the voice therapy mask covering said user's mouth and nose;
setting a positive end-expiratory pressure (PEEP) valve to an impedance level;
sealing the voice therapy mask against said user's face to form a seal capable of withstanding at least the impedance level; and
while the voice therapy mask is sealed against said user's face, performing a therapy protocol.

6. The method of claim 5, wherein the therapy protocol includes:
producing a first kind of vocalization in the voice therapy mask;
producing a second kind of vocalization in the voice therapy mask, the second kind of vocalization being different from the first kind of vocalization; and
producing natural language in the voice therapy mask.

7. The method of claim 5, wherein sealing the voice therapy mask includes sealing the voice therapy mask to an impedance level of at least 5 cm water.

8. The method of claim 5, further comprising using a hydration protocol to check user hydration prior to performing the therapy protocol.

9. A method for speech therapy, comprising:
providing a voice therapy mask to apply to a user's face, the voice therapy mask covering said user's mouth and nose and creating a seal such that exhaled air may only exit the voice therapy mask through a positive end-expiratory pressure (PEEP) valve connected to an opening in the voice therapy mask;
instructing said user to produce a first kind of vocalization in the voice therapy mask;
instructing said user to produce a second kind of vocalization in the voice therapy mask, the second kind of vocalization being different from the first kind of vocalization; and
instructing said user to produce natural language in the voice therapy mask.

10. The method of claim 9, wherein the voice therapy mask does not restrict a freedom of movement of said user's mouth to produce at least one of the first kind of vocalization, the second kind of vocalization, or the natural language.

11. The method of claim 9, wherein the first kind of vocalization is a vowel at a high pitch.

12. The method of claim 9, wherein the second kind of vocalization changes in pitch from low to high.

13. The method of claim 12, further comprising instructing said user to produce a third vocalization in the voice therapy mask, wherein the third vocalization changes in pitch from high to low.

14. The method of claim 13, wherein at least one of the first kind of vocalization or the second kind of vocalization is received twice.

15. The method of claim 9, wherein the natural language is received for at least three minutes.

16. The method of claim 9, wherein the natural language includes at least one of speaking, reading, or singing.

17. The method of claim 9, wherein the method is repeated twice per day.

18. The method of claim 9, further comprising using a hydration protocol to check user hydration prior to applying the voice therapy mask.

19. The method of claim 9, further comprising setting an impedance level at the PEEP valve.

20. The method of claim 19, further comprising changing the impedance level at the PEEP valve.
